(19) United States
(12) Patent Application Publication
(10) Pub. No.: US 2007/0196861 A1
(43) Pub. Date: Aug. 23, 2007

(54) METHOD FOR FORMING FILM, BASE PLATE WITH FILM, SENSOR, AND LIQUID COMPOSITION

(75) Inventors: Hitoshi FUKUSHIMA, Fujimi-cho (JP); Hiroshi TAKIGUCHI, Suwa-shi (JP)

Correspondence Address: OLIFF & BERRIDGE, PLC, P.O. BOX 19928, ALEXANDRIA, VA, 22320, US

(73) Assignee: SEIKO EPSON CORPORATION, Tokyo (JP)

(21) Appl. No.: 11/673,347

(22) Filed: Feb. 9, 2007

(30) Foreign Application Priority Data
Feb. 22, 2006 (JP) 2006-045937
Dec. 12, 2006 (JP) 2006-335122

(51) Int. Cl. G01N 33/53; C12M 3/00

(52) U.S. Cl. 435/7.1; 435/287.2; 427/2.11

(57) ABSTRACT

A method for forming a film comprises: preparing a liquid composition containing a substance for detecting an object and a polyether derivative having a weight-average molecular weight of 2000 or less; supplying the liquid composition to a side adjacent to a first surface of a base plate by using a droplet discharge method to form a liquid film; and drying the liquid film to form a detection film containing the substance.

### BACKGROUND

1. Technical Field

Several aspects of the present invention relate to a method for forming a film,, a base plate with a film, a sensor, and a liquid composition.

2. Related Art

Recently, biological reactions that involve protein are observed or detected in real time. For example, physiologically active protein, such as enzymes and antibodies, is observed in vitro while fixed on a base plate.

In order to observe the biological reactions more accurately, it is necessary that protein is supported on the base plate with high film forming accuracy. As a method for fixing protein on a base plate, an inkjet method is widely used in which a liquid containing protein is used as ink, supplied on a base plate. For example, the method is disclosed in JP-A-61-245051.

In the inkjet method, ink supplied in an ink chamber is discharged onto a base plate from a small hole provided at a side opposite to a vibrating plate by changing the volume of the ink chamber (space) with the vibrating plate vibrated by a piezoelectric element.

The inkjet method needs to realize a stable and high accurate discharging amount of a liquid discharged from the small hole, high accurate landing position of a droplet discharged from the small hole, few case of clogging in the small hole, and so on. In order to satisfy the demands, viscosity and surface tension of ink (a liquid containing protein) is adjusted,

As a method for adjusting viscosity and surface tension of ink, a polyether derivative is added in ink, for example.

However, fixing protein on a base plate using such ink has a problem that the physiological activity of protein is lowered (lost activity) due to a contact between the polyether derivative and protein contained in the ink.

Such problem may occur when a film containing nucleic acid, fluorescent dye and the like is formed on a base plate using an inkjet method, in similar way of protein.

### SUMMARY

An advantage of the invention is to provide a method for forming a film, which method can form a detection film containing a detection substance with favorable accuracy and suppress or prevent the activity of the detection substance in the detection film from being lowered, a base plate with a film, which base plate is provided with the detection film formed by the method, and a sensor having high reliability. The advantage is also to provide a liquid composition that can prevent or suppress the physiological activity of a detection substance contained in a detection film to be formed from being lowered, and form the detection film with favorable accuracy.

According to a first aspect of the invention, a method for forming a film includes: a first step to prepare a liquid composition containing a detection substance for detecting a detection object and a polyether derivative having a weight-average molecular weight of 2000 or less; a second step to supply the liquid composition to a side adjacent to a surface of a base plate by using a droplet discharge method to form a liquid film; and a third step to dry the liquid film to form a detection film.

The method can form a detection film that contains a detection substance with favorable accuracy, and suppress or prevent the physiological activity of the detection substance in the detection film from being lowered,

That is, the detection film can be formed with the detection substance improved in stability in the detection film by adding a polyether derivative having a weight-average molecular weight of 2000 or less into the detection film in addition to the detection substance,

The polyether derivative having a weight-average molecular weight of 2000 or less also has high affinity to a substance having a polar group, such as protein, and nucleic acid. Thus, the method is effective to a case where a substance having a polar group is used as the detection substance since the method forms the detection film by using the liquid composition containing the detection substance and the polyether derivative having a weight-average molecular weight of 2000 or less.

In the method, it is preferable that A divided by B be from 9 to 10⁵ where a content of the polyether derivative in the liquid composition is A wt % while a content of the substance in the liquid compound is B wt %.

The resulting relation allows the attaching rate of the polyether derivative to the detection substance to be set within a preferable range, and the physiological activity of the detection substance to be preferably suppressed or prevented from being lowered.

In the method, the liquid composition preferably includes 10 wt % or less of the detection substance.

The detection substance having a content within the above range can react thoroughly in a formed detection film.

In the method, the liquid composition preferably includes 90 wt % or more of the polyether derivative.

If the content of the polyether derivative is excess, viscosity of the liquid composition increases depending on the type of polyether derivative. There may be a case where the liquid composition could not be discharged as droplets by a droplet discharge method. If the content of the polyether derivative is too small, the liquid composition may be easily dried. In addition, with the content of the polyether derivative set within the above range, the attaching rate of the polyether derivative to the detection substance is set within more preferable range, allowing the lowering of the geological activity of the detection substance to be more preferably suppressed or prevented.

In the method, viscosity of the liquid composition is preferably 20 cP or less at normal temperature.

Within the above viscosity range, the liquid composition can be stably discharged as droplets by a droplet discharge method.

In the method, surface tension of the liquid composition is preferably from 20 mN/m to 70 mN/m at normal temperature.

Within the above surface tension range, a liquid film can be formed in an even thickness with droplets discharged by a droplet discharge method.

In the method, a variation of the polyether derivative is preferably within a range of ±0.2 C where C is a weight-average molecular weight of the polyether derivative.

In the above range, the molecular weight of the polyether derivative that comes into contact with the detection substance is uniformed, enabling the probability of the detection substance contacting with the polyether derivative having a large molecular weight to be reduced, As a result, the lowering of the physiological activity of the detection substance due to the contact with the one having a large molecular weight can be more preferably prevented or suppressed.

In the method, the polyether derivative preferably includes a main chain made of polyether and a functional group that is provided at least one end of the main chain and is capable of bonding the substance.

The functional group and the detection substance bond each other in the liquid composition when the detection substance and the polyether derivative contact. As a result the polyether derivative and the detection substance are joined. The joining improves adhesiveness between the detection substance and the polyether derivative. Consequently, the stability of the detection substance is further improved and the lowering of the physiological activity can more preferably be suppressed or prevented.

In the method, the main chain preferably has the functional group at both ends thereof.

This structure enhances the adhesiveness between the detection substance and the polyether derivative, allowing the lowering of the physiological activity to be more preferably suppressed or prevented.

In the first step of the method, the polyether derivative and the substance are preferably joined by bonding the substance and the functional group.

The joining enhances adhesiveness between the detection substance and the polyether derivative. Consequently, the stability of the detection substance is further improved and the lowering of the physiological activity can be more preferably suppressed or prevented.

In the method, the polyether derivative preferably mainly contains a polyethylene glycol derivative.

As a result, viscosity and surface tension of a liquid composition can be easily set within a preferable range.

In the method, the detection substance is preferably protein.

When the detection substance is protein, the lowering of the physiological activity of the detection substance can be more markedly suppressed or prevented by applying the method.

In the method, the detection substance is preferably an enzyme.

The reaction between the enzyme and the substrate can progress more stably and in a good reproducible fashion.

According to a second aspect of the invention, a base plate with a film is provided with a detection film formed at the side adjacent to the surface of the base plate by the method of the first aspect.

The base plate can be provided with the detection film in which the lowering of the physiological activity of the detection substance contained therein is suppressed or prevented, and that is formed with favorable film forming accuracy.

According to a third aspect of the invention, a base plate with a film includes a detection film containing a detection substance for detecting a detection object.

The detection film includes the detection substance and a polyether derivative having a weight-average molecular weight of 2000 or less.

The base plate can be provided with the detection film in which the lowering of the physiological activity of the detection substance contained therein is suppressed or prevented, and that is formed with favorable film forming accuracy.

According to a fourth aspect of the invention, a sensor is provided with the base plate of the third aspect.

This structure allows a highly reliable sensor to be provided.

When the polyether derivative having a weight-average molecular weight of 2000 or less is applied to a sensor in which a detection electrode is provided on the base plate, and an electrical signal is obtained by receiving electrons between the detection electrode and the detection substance or detection object detected by the detection substance by using the detection electrode, the distance between the detection electrode and the detection substance can be set to an appropriate distance for getting the electrical signal.

According to a fifth aspect of the invention, a liquid composition includes: a detection substance having a physiological activity; and a polyether derivative having a weight-average molecular weight of 2000 or less.

The liquid composition can prevent or suppress the lowering of the physiological activity of a detection substance contained in a detection film to be formed, and form the detection film with favorable accuracy.

### BRIEF DESCRIPTION OF THE DRAWINGS

The invention will be described with reference to the accompanying drawings, wherein like numbers reference like elements.

FIG. 1 is a longitudinal cross-sectional view schematically illustrating the structure of a sensor of an embodiment of the invention.

FIG. 2 is a schematic view illustrating a method for manufacturing the sensor shown in FIG. 1.

### DESCRIPTION OF EXEMPLARY EMBODIMENTS

A method for forming a film, a base plate with a film, a sensor, and a liquid composition according to the invention will now be described in detail based on preferred embodiments with reference to the accompanying drawings.

Sensor

First, a sensor according to an embodiment of the invention will be described.

In the embodiment, a sensor is exemplified that is provided with an enzyme electrode layer containing physiologically active protein as a detection film containing a detection substance.

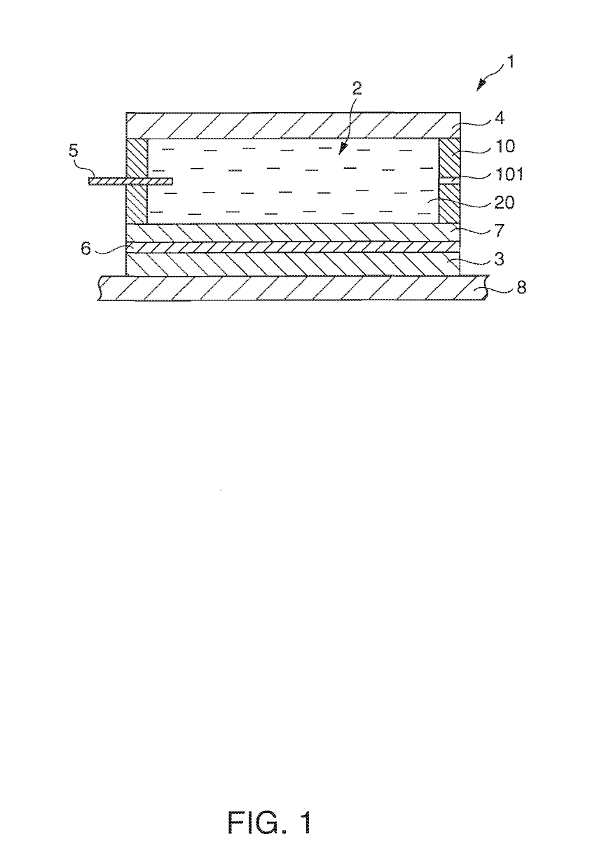

FIG. 1 is a longitudinal cross-sectional view schematically illustrating the structure of the sensor of the embodiment. In addition, in the following description, the top side in FIG. 1 is described as “up,” while the bottom side is described as “down”.

As shown in FIG. 1, a sensor 1 is provided with a base plate 8, on which a sample store space 2 storing a liquid sample 20, a working electrode 3, a counter electrode 4, a reference electrode 5, an under layer 6, and an enzyme electrode layer 7 are structured in layers. The sensor 1 detects the quantity of a target (detection object) 21 in the liquid sample 20 supplied in the sample store space 2 as a value of current that flows when a voltage is applied between a pair of electrodes 3 and 4, provided on the base plate 8 in a plurality of numbers.

In the embodiment, a base plate with a film according to the invention is exemplified as a structure composed of the base plate 8 and the enzyme electrode layer 7 formed above the base plate 8. In other words, in the embodiment, the enzyme electrode layer 7 is formed by a method for forming a film according to the invention, which will be minutely described later in a method for manufacturing a sensor.

Examples of the liquid sample 20 include body fluids such as blood, urine, sweat, lymph, liquor cerebrospinalis, bile, and saliva, and processed liquids that these body fluids have been subjected to various processes.

The target emits electrons (e⁻) by reacting with protein, which will be described later.

Examples of the target include sugars such as glucose, proteins such as simple protein and glycoprotein, alcohols, steroids such as cholesterol, steroid hormones, bile acid, and bile alcohol, vitamins, hormones, lactic acid, bilirubin, uric acid, and creatinine.

The base plate 8 supports each part included in the sensor 1.

As the base plate 8, one or more than one in mixture of the following exemplary materials can be used: resin materials such as polyethylene, polypropylene, polystyrene, polyethylene terephthalate (PET, polyacrylic, and epoxy resins, glass materials; and ceramics materials. When composite materials are used as the material for the base plate 8, nonflammable printed boards made of composite materials of glass fibers and epoxy resin can be used.

On the base plate 8, the working electrode 3 is individually provided in a plurality of numbers.

Examples of material for each of the working electrode 3, counter electrode 4, and the reference electrode 5 include metal materials such as gold, silver, copper, platinum, and alloys thereof; metal oxide materials such as ITO, and carbon materials such as carbon.

Above the working electrode 3, the enzyme electrode layer 7 is disposed with the under layer (Intermediate layer) 6 interposed therebetween.

As shown in FIG. 1, the enzyme electrode layer 7 is provided so as to expose at least a part of thereof (in the embodiment, the upper surface) to the sample store space 2, and contains protein having physiological activity (hereinafter, simply referred to as protein), in which protein reacts with the target with electrons (e⁻) emitted.

Examples of protein include polypeptide and oligopeptide like enzymes or antibodies. Among them, enzymes are preferable. This allows electrons (e⁻) to be emitted stably and in a good reproducible fashion in the reaction between protein (enzyme) and the target (substrate). That is, the reaction between the enzyme and the substrate can progress more stably and in a good reproducible fashion.

The enzyme is appropriately chosen in accordance with the kind of target to be measured. For example, oxidases and dehydrogenases can be used.

Examples of oxidase include glucose oxidase, galactose oxidase, pyruvate oxidase, D- or L-amino acid oxidase, amine oxidase, cholesterol oxidase, and choline oxidase.

Examples of dehydrogenase include alcohol dehydrogenase, glutamic acid dehydrogenase, cholesterol dehydrogenase, aldehyde dehydrogenase, glucose dehydrogenase, fructose dehydrogenase, sorbitol dehydrogenase, and glycerol dehydrogenase.

In addition, the enzyme electrode layer 7 contains a polyether derivative having a weight-average molecular weight of 2000 or less. The enzyme electrode layer 7 in the sensor of the embodiment is an exemplary film formed by a method for forming a film according to the invention. Effects brought about by containing a polyether derivative having an average weight molecular weight of 2000 or less in the enzyme electrode layer 7 will be minutely described in a method for manufacturing the sensor 1.

The enzyme electrode layer 7 preferably contains a mediator (intermediary substance) that carries emitted electrons to the under layer 6. As a result, electrons can be efficiently transferred to the working electrode 3 from the enzyme electrode layer 7 through the under layer 6 since the mediator plays a role of transferring electrons.

As the mediator, one or more than one in mixture of the following exemplary substances can be used: ferrocene and its derivatives; potassium ferricyanide; nickelocene and its derivatives; quinone and its derivatives such as p-benzoquinone, and pyrrolo quinoline quinone; flavin derivatives such as flavin adenine dinucleotide (FAD); nicotinamide derivatives such as nicotinamide adenine dinucleotide (NAD) and nicotinamide adenine dinucleotide phosphate (NADP); phenazine methosulfate; 2,6-dichlorophenol indophenol; hexacyanoferrate (III); and octacyanotungsten ion.

The under layer 6 is provided between the working electrode 3 and the enzyme electrode layer 7 to come in contact with the both of them. The under layer 6 reliably prevents the working electrode 3 and the enzyme electrode layer 7 or the constituent material of the working electrode 3 and protein from being directly come into contact with each other. As a result, the alteration and deterioration of protein due to the contact with the constituent material of the working electrode 3 or the lowering of the physiological activity of protein (lost activity) can be preferably suppressed or prevented.

As the material for the under layer 6, one or more than one in mixture of the following exemplary materials can be used: natural polymers such as biologicalally-relevant polymers (animal-derived polymers) and plant-derived polymers; synthetic polymers (synthetic resin); and modified thereof. Among them, one mainly containing biologicalally-derived polymers is preferable. The under layer 6 made of the above material can realize that electrons are reliably transferred from the enzyme electrode layer 7 to the working electrode 3 through the under layer 6, and preferably prevent the physiological activity of protein from being lost.

Examples of such biologicalally-relevant polymers include albumin (e.g. bovine serum albumin: BSA), globulin, and myoglobin. Materials mainly contain such biologicalally-relevant polymers are preferably used for the under layer 6. Examples of the materials include a mixed polymer of carboxymethylcellulose and BSA, a mixed polymer of polyvinyl pyrrolidone and BSA, and a mixed polymer of polyethylene glycol and BSA.

Here, the under layer 6 can be omitted if the lowering of the physiological activity of protein can be suppressed or prevented even though the constituent material of the working electrode 3 and protein are come in contact with each other depending on their combinations.

The counter electrode 4 is disposed so as to face the working electrode 3 and expose at least a part thereof (the lower surface, in the embodiment) to the sample store space 2.

Between the counter electrode 4 and the working electrode 3, a voltage is applied. When a voltage is applied between the working electrode 3 as a positive potential and the counter electrode as a negative potential with the liquid sample 20 supplied in the sample store space 2, electrons that are generated and emitted by a reaction between the target and protein move to the working electrode 3. As a result, the working electrode 3 and the counter electrode 4 are electrically conducted.

The voltage applied between the working electrode 3 and the counter electrode 4 is preferably 1.0 V or less, more preferably from about 0.1 V to about 0.5 V. Applying a voltage within the above range allows between the working electrode 3 and the counter electrode 4 to be especially fast and reliably conducted. In addition, the occurrence of electrons due to electrolysis of water can be reliably prevented (avoided).

The reference electrode 5 is fixed to a sealing portion (partition wall) 10 so that a part of the electrode 5 is placed in the sample store space 2. The reference electrode 5 provides (sets) a standard potential with respect to the working electrode 3. Setting the standard potential by providing the reference electrode 5 can improve the measuring accuracy and the repeatability of a target quantity. That is, variations in measured values can be reduced when various sensors and the liquid samples are used.

The sensor 1 is used for detecting the target quantity in the liquid sample 20 as aforementioned. For example, it is applied to monitor a blood sugar count of diabetic patients.

In the sensor 1 used in the above case, when the liquid sample 20 is supplied into the sample store space 9, a target contained in the liquid sample 20 diffuses in the enzyme electrode layer 7. The target diffused in the enzyme electrode layer 7 reacts with protein, electrons being emitted.

In a case where the target is glucose and protein is glucose oxidase, as an example, glucose is oxidized by catalysis of glucose oxidase, and is decomposed to gluconic acid with electrons produced and emitted as shown in formula (1).

C₆H₁₀O₆+2H₂O+2O₂→C₆H₈O₆+3H₂O₂→C₆H₈O₆3O₂+6H⁺+6e⁻ formula (1)

When the mediator is potassium ferricyanide, a ferricyanide ion is reduced to a ferrocyanide ion as shown in formula (2) by electrons produced as expressed in formula (1).

[Fe(III)(CN)₆]³⁻+e⁻→[Fe(II)(CN)₆]⁴⁻ formula (2)

In this case, a ferrocyanide ion is oxidized to a ferricyanide ion in the vicinity of the working electrode 3 if a voltage is applied between the working electrode 3 as a positive potential and the counter electrode 4 as a negative potential. As a result, an electron flow or current occurs between the electrodes 3 and 4.

Therefore, the target quantity contained in the liquid sample 20 can be indirectly measured by detecting the value of current flowed between the electrodes 3 and 4 when a given voltage is applied between the electrodes.

Specifically, the target quantity is obtained by the following procedures. A plurality of samples having a known target quantity is prepared. Each value of the current flowed between the working electrode 3 and the counter electrode 4 is measured by using each sample to obtain a relationship between the target quantity and the measured current value. That is, a standard curve or table is made. Then, a value of current flowed between the working electrode 3 and the counter electrode 4 is measured by using the liquid sample 20 subjected to actual measurement. The measured value is converted to a target amount based on the preliminary prepared standard curve or table. As a result, the target quantity contained in the liquid sample 20 can be determined.

Usually, a program for conducting these procedures is installed in a reader (measuring equipment). This program allows measurers to know the target quantity only by attaching the sensor 1 to the reader and supplying the liquid sample 20 into the sample store space 2 of the sensor 1.

As described above, the sensor 1 can measure the target quantity contained in the liquid sample 20 by detecting (measuring) the electrons emitted by the reaction of the target and protein as the value of current flowed between the working electrode 3 and the counter electrode 4.

In the embodiment, the sensor I is described that detects the reaction of the target and protein as a current value and the target quantity based on the current value. However, the sensor according to the invention may employ a method for optically detecting a target amount, in which method a reagent showing color reactions is used and the reaction of the target and protein is indirectly measured by observing the absorbance change of the reagent, for example.

Method for Manufacturing a Sensor

The sensor 1 can be manufactured by the following manner, for example.

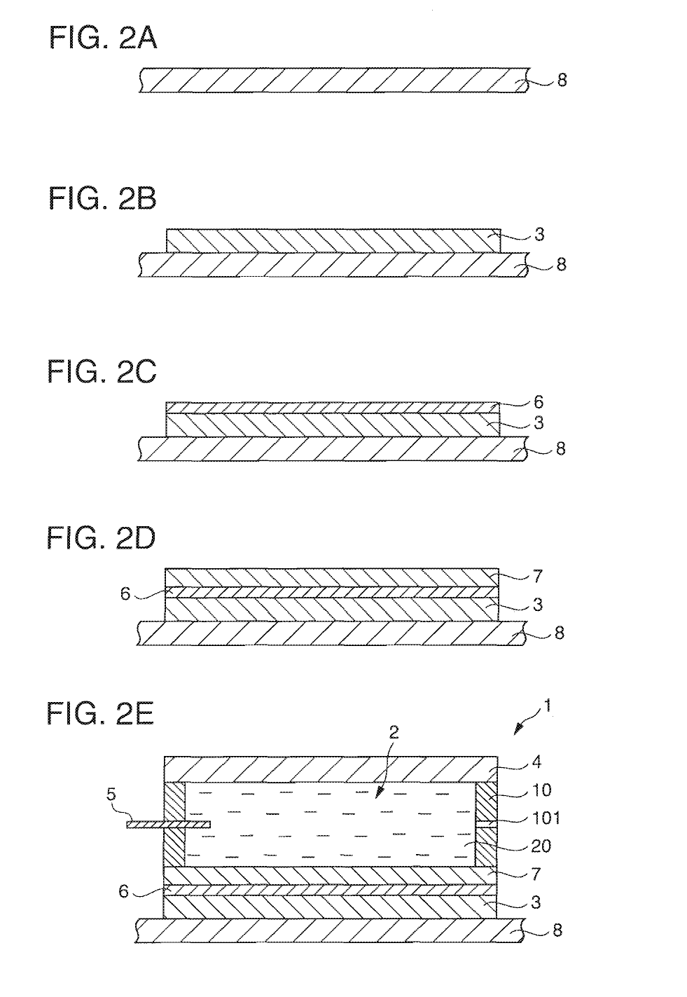

FIGS. 2A through 2D are longitudinal sectional views illustrating a method for manufacturing the sensor shown in FIG. 1. In the following description, the top side in FIG. 2 is described as “up,” while the bottom side is described as “down”.

[1] First, the base plate 8 is prepared as shown in FIG. 2A.

Then, the working electrode 3 is formed on the base plate 8 as shown in FIG. 2B. The working electrode 3 can be formed by the following manner, for example.

First, a metal film (a metal layer) is formed so as to cover the upper surface (electrode forming surface) of the base plate 8. This formation can be achieved by the following exemplary methods: chemical vapor deposition (CVD) such as plasma CVD, thermal CVD, and laser CVD; dry plating such as ion plating, vacuum deposition, and sputtering; wet plating such as electrolytic plating, immersion plating, and electroless plating; thermal spraying; MOD methods; and metal foil bonding.

Then, a resist material is coated (supplied) on the metal film and cured, forming a resist layer having a shape corresponding to the shape of the working electrode 3.

Next, unnecessary portions of the metal film are removed by using the resist layer as a mask. This metal film can be removed by one or more than one in mixture of the following exemplary methods; physical etching such as plasma etching, reactive ion etching, beam etching, and photo assist etching; and chemical etching such as wet etching.

Then, the working electrode 3 can be achieved by removing the resist layer.

The working electrode 3 can also be formed by coating (supplying) a conductive material including conductive particles on the base plate 8, and then carrying out a post-process (e.g., heating, irradiation with infrared rays, or applying ultrasonic waves) on the coated film if needed.

The coating can be conducted by one or more than one in mixture of the following exemplary methods: a spin-coating method, a casting method, a micro gravure coating method, a gravure coating method, a bar coating method, a roll coating method, a wire bar coating method, a dip coating method, a spray coating method, a screen printing method, a flexographic printing method, an offset printing method, an inkjet method, and a micro-contact printing method. Among them, the inkjet method is especially preferable as the coating method. Using the inkjet method allows the conductive material to be supplied to a desired area of the base plate 8, enabling the working electrode 3 to be formed in a desired shape without forming the resist layer as described above.

[2] Next, as shown in FIG. 2C, the under layer 6 is formed on the working electrode 3.

When the under layer 6 is structured by using the biologicalally-derived polymers, the under layer 6 can be formed by the following manner.

First, a liquid material is prepared by dissolving a biologicalally-derived polymer into a solvent.

Examples of the solvent include: inorganic solvents such as phosphoric acid, nitric acid, sulfuric acid, ammonia, hydrogen peroxide, and water; amid solvents such as N,N-dimethylformamide (DMF), N,N-dimethylacetamide (DMA), and N-methyl-2-pyrrolidone (NMP); sulfuric compound solvents such as dimethyl sulfoxide (DMSO), and sulfolane; and mixed solvents thereof.

The concentration (content) of the biologicalally-derived polymer in the liquid material is preferably 20 wt % or less, more preferably from about 1 wt % to about 10 wt %.

Next, the prepared liquid material is supplied to the working electrode 3, and then dried. As the method for supplying the liquid material to the working electrode 3, the inkjet method is especially preferable, for example, among the above coating methods.

[3] Next, as shown in FIG. 2D, the enzyme electrode layer 7 is formed on the under layer 6.

Since the enzyme electrode layer 7 contains protein therein (film) and is formed corresponding to the shape of the under layer 6, a method for forming a film according to the invention is applied to form the enzyme electrode layer 7.

Here, a method that supplies a liquid composition containing protein to a predetermined area by using an inkjet method is preferably used when a film such as the enzyme electrode layer 7 is selectively formed on a predetermined area such as on the under layer 6.

However, as described in the background, forming a film by using an inkjet method has a problem that the physiological activity of protein is lowered (lost activity) due to the contact of protein with a polyether derivative, which is contained in order to adjust viscosity or surface tension of ink (liquid compound).

As a result of the dedicated investigations in view of the above problem, the inventor found that the lost activity of protein was closely related to the weight-average molecular weight of a polyether derivative with which protein contacts.

As a result of the further investigations, the inventor found that the lowering of the physiological activity could be preferably prevented or suppressed by setting the weight-average molecular weight of the polyether derivative to 2000 or less, improving the stability of protein.

A method for forming the enzyme electrode layer 7 will be minutely described below by applying a method for forming a film according to the invention.

[3-1] First, a liquid composition (an example of the liquid composition of the invention) is prepared that contains protein and a polyether derivative having a weight-average molecular weight of 2000 or less (a first process).

Examples of the solvent used in preparing the liquid composition include: inorganic solvents such as phosphoric acid, nitric acid, sulfuric acid, ammonia, hydrogen peroxide, and water; amid solvents such as N,N-dimethylformamide (DMF), N,N-dimethylacetamide (DMA), and N-methyl-2-pyrrolidone (NMP); sulfuric compound solvents such as dimethyl sulfoxide (DMSO), and sulfolane; and mixed solvents thereof.

The polyether derivative is contained in the liquid composition in order to adjust viscosity or surface tension of the liquid composition so as to form the enzyme electrode layer 7 on the under layer 6 with high film forming accuracy.

The weight-average molecular weight of the polyether derivative is preferably set to 2000 or less, more preferably from about 300 to about 1000. The weight-average molecular weight set within above range can set viscosity or surface tension of the liquid composition within a preferable range. In addition, the polyether derivative hardly acts as a steric barrier to the active portion of protein when the polyether derivative comes into contact with protein. Therefore, the stability of protein is improved, preferably maintaining the physiological activity.

Further, the variation of the polyether derivative is preferably within the range of ±0.2 C, more preferably within the range of ±0.1 C, where C is the weight-average molecular weight of the polyether derivative. The molecular weight of the polyether derivative that comes into contact with protein is uniformed, enabling the probability of protein contacting with the polyether derivative having a large molecular weight to be reduced. As a result, the lowering of the physiological activity of protein due to the contact with the one having a large molecular weight can be prevented or suppressed.

The polyether derivative also preferably includes a main chain made of polyether and a functional group capable of bonding protein (hereinafter, simply referred to as a functional group) at at least one of the sides of the main chain. The functional group and protein bond each other in the liquid composition when protein and the polyether derivative contact. As a result the polyether derivative joins protein. The joining improves adhesiveness between protein and the polyether derivative. Consequently, the stability of protein is further improved and the lowering of the physiological activity can more preferably be suppressed or prevented.

The polyether derivative more preferably includes the functional group at both ends. As a result, adhesiveness between protein and the polyether derivative is further improved, performing the above effect more notably.

Specifically, examples of the functional group are materials that include the followings at end thereof an amino group; a carboxyl group; N-hydroxy succinimide ether (NHS) group; a thiol group; a maleimide group; a halogen group; and a disulfide group shown in chemical formula (1).

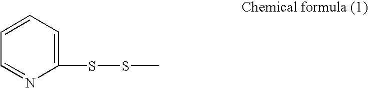

For example, the amino group and the carboxyl group included in protein form an amide bond, resulting in protein and the polyether derivative being bonded. The carboxyl group and the amino group included in protein form an amide bond, resulting in protein and the polyether derivative being bonded. The amino group included in protein is reacted with the N-hydroxy succinimide ether group, resulting in protein and the polyether derivative being bonded. The thiol group included in protein is reacted with the functional group expressed by chemical formula (1), resulting in protein and the polyether derivative being bonded.

The polyether derivative may be applicable in which, for example, at least one of the ends thereof is substituted with a substitution group that does not bond protein, or the ends are not substituted with a substitution group, other than one in which at least one of the ends thereof is substituted with the substitution group capable of bonding protein as described above.

Examples of such substitution group include: a ring hydrocarbon group such as an alkyl group, a phenyl group, a biphenyl group, and a cyclohexane group; and a hydroxyl group.

As the main chain made of polyether, one or more than one in mixture of the following exemplary substances can be used: polyethylene glycol, polypropylene glycol, and polymethylene glycol. Among them, one mainly made of polyethylene glycol is preferable. In other words, the polyether derivative preferably mainly contains a polyethylene glycol derivative. As a result, viscosity and surface tension of a liquid composition can be easily set within a preferable range.

As a specific example of the polyether derivatives, a polyethylene glycol derivative expressed in chemical formula (2) is exemplified.

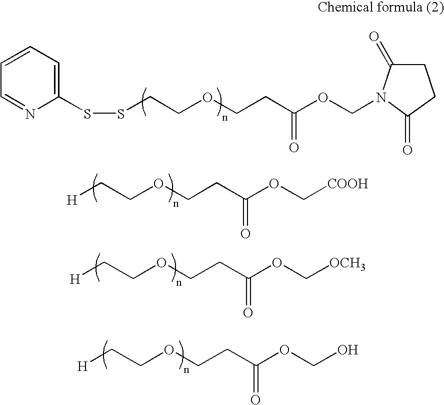

Where n is an integer of one or more. For example, n is set from about 5 to about 20 when the weight-average molecular weight of the polyethylene glycol derivative is from about 300 to about 1000.

The content A (wt %) of the polyether derivative in the liquid composition and the content B (wt %) of protein preferably satisfies the relation that A/B is from 9 to 10⁵, more preferably from 20 to 10⁴. With the contents of the polyether derivative and protein satisfying the above relation, the attaching rate of the polyether derivative to protein is set within a preferable range, preferably suppressing or preventing the lowering of the physiological activity of protein.

The content of protein in the liquid composition is preferably 10 wt % or less, more preferablv from about 0.01 wt % to about 5 wt %. The content allows protein to thoroughly react with the target contained in the liquid sample 20 in the enzyme electrode layer 7. As a result, electrons can be emitted efficiently.

The content of the polyether derivative in the liquid composition is preferably 90 wt % or more, more preferably from about 95 wt % to about 99.9 wt %. If the content of the polyether derivative is excess, viscosity of the liquid composition increases depending on the type of polyether derivative. There may be a case where the liquid composition could not be discharged as droplets by an inkjet method. If the content of the polyether derivative is too small, the liquid composition may be easily dried. In addition, with the content of the polyether derivative set within the above range, the attaching rate of the polyether derivative to protein is set within more preferable range, more preferably suppressing or preventing the lowering of the geological activity of protein.

Here, the mediator or the like is added into the liquid composition, if necessary.

When the mediator is included in the liquid composition, its content is preferably from about 0.1 wt % to about 1 wt %, more preferably about 0.1 wt %. The mediator of the above content allows electrons emitted from the target in the enzyme electrode layer 7 formed in a later step [3-3] to reliably and rapidly move to the working electrode 3 through the under layer 6.

The viscosity of the liquid composition (ink) is preferably, but not limited to, about 20 cP or less at normal temperature, and is more preferably from about 4 cP to about 10 cP. Within the viscosity range, the liquid composition can be stably discharged as droplets by an inkjet method.

The surface tension of the solvent is preferably, but not limited to, from about 20 mN/m to about 70 mN/m in general, and is more preferably from about 30 mN/m to about 50 mN/m. Within the surface tension, a liquid film can be formed on the under layer 6 in an even thickness with droplets discharged by an inkjet method.

[3-2] Next, the liquid composition is supplied on the under layer 6 (a side adjacent to one surface) by using an inkjet method to form a liquid film (a second step).

With the viscosity and surface tension of the liquid composition set within the above range, the liquid composition can be stably discharged (supplied) on the under layer 6 as droplets, and a liquid film can be formed in an even thickness.

[3-3] Next, the liquid film formed on the under layer 6 is dried to form the enzyme electrode layer 7 (a third step).

In the embodiment, the enzyme electrode layer 7 in which the polyether derivative having a weight-average molecular weight of 2000 or less is applied to the sensor 1 in which an electrical signal is obtained by electrons that are produced by the reaction between the target and the protein, and move from the target to the working electrode 3 via a mediator. In this case, the distance between the working electrode 3 and protein or the target is set appropriate for getting the electrical signal.

The surrounding temperature in drying the liquid film is preferably from about 20° C. to about 80° C., more preferably from about 20° C. to about 45° C.

The drying time is preferably from about 10 minutes to about 150 minutes, more preferably from about 30 minutes to 100 minutes.

With the condition of drying the liquid film set within the above range, the lowering the physiological activity of protein contained in the liquid film can be reliably prevented., and the liquid film can be reliably and rapidly dried to form the enzyme electrode layer 7 having an even thickness.

When the polyether derivative has a functional group at ends thereof, protein contained in the liquid film and the functional group bond more reliably by setting the condition of drying the liquid film within the range described above. As a result, the bonding (linking) rate of the polyether derivative to protein can be more reliably improved.

Through the above steps, the enzyme electrode layer 7 having favorable film forming accuracy can be formed on the under layer 6, with the lowering of the physiological activity of protein preferably suppressed or prevented.

The polyether derivative having a weight-average molecular weight of 2000 or less has high affinity to substances having a polar group, such as nucleic acid, and protein having physiological activity, such as enzymes. Therefore, when an enzyme is used as a detection material as shown in the embodiment, forming the detection film by using the method for forming a film according to the invention is especially effective.

In the embodiment, a method for supplying a liquid material as a droplet to a predetermined area, i.e., an inkjet method is described as a droplet discharge method. However, the method is not limited to this, a bubble jet (bubble jet is a registered trade mark) method may be used as the droplet discharge method, for example.

[4] Next, as shown in FIG. 2E, the counter electrode 4 is disposed so as to face the working electrode 3 while the reference electrode 5 is disposed to a predetermined position. Then an adhesive is supplied on the periphery while the electrodes 3 and 5 are disposed, and cured to form the sealing portion 10. As a result, the sample store space 2 is defined by the enzyme electrode layer 7, the counter electrode 4, and the sealing portion 10.

Examples of the adhesive include an epoxy adhesive, an acrylic adhesive, and a rubber adhesive.

In this case, an injection opening (inlet) 101 to supply the liquid sample 20 into the sample store space 2 is formed in the sealing portion 10.

Through the above steps, the sensor 1 shown in FIG. 1 is completed.

A method for forming a film, a base plate with a film, a sensor, and a liquid composition according to the invention are not limited to the above embodiments described using the accompany drawings.

For example, the method for forming a film of the invention may include one or more additional steps for any purpose.

The base plate with a film of the invention can be applied to protein microarrays in which a physiologically active protein is supported on a base plate, gene analysis chips and the like, in addition to the above sensor.

Each element of the sensor of the invention may be replaced with any other structures having similar functions.

For example, one or more than one layer may be provided between layers for any purpose (to improve adherence) in a range without lowering the characteristics of the sensor.

In the embodiment, a, sensor is exemplarily described in which physiologically active protein is used as a detection substance and electrons produced in protein reacting with a target are detected. However, it is not limited to this case.

For example, ones excluding protein having physiological activity, and ones excluding protein not having physiological activity can be used as the detection substances. Here, physiological activity means catalystic function of enzyme.

Here, examples of ones excluding protein having physiological activity include nucleic acid such as DNA and RNA, and steroid hormone.

As ones excluding protein not having physiological activity, fluorescent dye is exemplified.

Even though the above substances are used as the detection substance, the same effect of the physiologically active protein used can be achieved. Obviously, a detection film is preferably formed by using physiologically active protein as a detection substance since the above-described effect can be markedly achieved.

### EXAMPLES

Specific examples of the invention will now be described.

1. Preparing a Liquid Composition

Sample No. 1

A liquid composition was prepared by dissolving glucose oxidase (300 units/mg: protein), the polyethylene glycol derivative (polyether derivative) expressed by chemical formula (3), and ferrocene (mediator) into a phosphate buffer solution (10 mH:pH7.8).

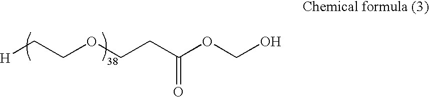

The liquid composition was prepared so that each material has the following respective concentrations in the liquid composition. Protein: 100 units/mL Polyethylene glycol derivative: 1.0 mmol/mL Mediator: 0.01 mmol/mL.

Sample No. 2

The liquid composition was prepared in the same manner as the sample No. 1, except that the polyethylene glycol derivative expressed by chemical formula (4) was used instead of the polyethylene glycol derivative expressed by chemical formula (3) as the polyether derivative.

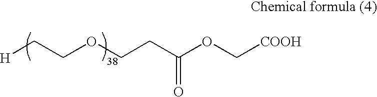

Sample No. 3

The liquid composition was prepared in the same manner as the sample No. 1, except that the polyethylene glycol derivative expressed by chemical formula (5) was used instead of the polyethylene glycol derivative expressed by chemical formula (3) as the polyether derivative.

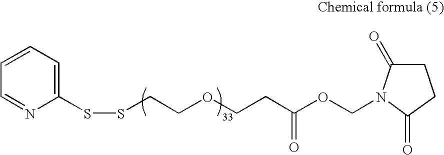

Sample No. 4

The liquid composition was prepared in the same manner as the sample No. 1, except that the polyethylene glycol derivative expressed by chemical formula (6) was used instead of the polyethylene glycol derivative expressed by chemical formula (3) as the polyether derivative.

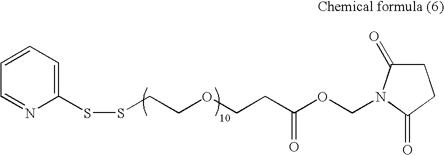

Sample No. 5

The liquid composition was prepared in the same manner as the sample No. 1, except that the polyethylene glycol derivative expressed by chemical formula (7) was used instead of the polyethylene glycol derivative expressed by chemical formula (3) as the polyether derivative.

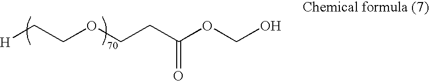

Sample No. 6

The liquid composition was prepared in the same manner as the sample No. 1, except that the polyether derivative and the mediator were not added.

2. Making Sensors

Sensors were manufactured in the following examples and comparative example by 5 pieces per each example.

### Example 1

Step 1. First, a glass base plate was prepared, and then a silver (Ag) electrode (working electrode) having an average thickness of 200 nm was formed on it by vacuum evaporation.

Step 2. Next, a solution containing albumin was prepared by dissolving albumin as a biologicalally-relevant polymer into purified water.

Then, the solution containing albumin was supplied on the working electrode by using an inkjet method to form a liquid film. The film was dried at 60° C. for 30 minutes to achieve an under layer.

Step 3. Next, the liquid composition of sample No. 1 was supplied into an ink chamber provided in the inkjet printer (PM700 available from Seiko Epson Corp.). Then, the liquid composition was applied on the under layer as droplets by operating piezoelectric elements equipped to the ink chamber. As a result, a liquid film was formed on the under layer. The viscosity and surface tension of the liquid composition were 3.6 cP and 32 mN/m respectively. The piezoelectric elements were vibrated by the following conditions. Driving voltage: 28 V. Frequency: 10 KHz.

Next, the liquid film was dried at 50° C. for 40 minutes in the atmospheric pressure, so that an enzyme electrode layer having an average thickness of 50 nm was formed on the under layer.

Step 4. Next, a counter electrode made of platinum was disposed so as to face the working electrode. A reference electrode made of carbon was disposed at a predetermined position. Then, an epoxy adhesive was supplied to the periphery and cured to form a sealing portion with the electrodes disposed. As a result, a sample store space was defined by an active layer, the counter electrode, and the sealing portion.

In this regard, an injection opening to supply a liquid sample into the sample store space was formed in the sealing portion.

Consequently, the sensors were completed.

### Example 2

Sensors were made in the same manner of the example 1, except that the sample No. 2 was used as the liquid composition instead of the sample 1 in the step 3.

### Example 3

Sensors were made in the same manner of the example 1, except that the sample No. 3 was used as the liquid composition instead of the sample 1 in the step 3.

### Example 4

Sensors were made in the same manner of the example 1, except that the sample No. 4 was used as the liquid composition instead of the sample 1 in the step 3.

### Comparative Example

Sensors were made in the same manner of the example 1, except that the sample No. 5 was used as the liquid composition instead of the sample 1 in the step 3.

3. Evaluation

3-1. Evaluation on Liquid Compositions

An evaluation reagent containing horseradish peroxidase (HRP), 4-aminoantipyrine (4-AA), and phenol was added into the liquid composition of 1 mL taking from each sample number.

The evaluation reagent was prepared so that the concentrations of HRP, 4-AA, and phenol in the reagent were 100 units/,mL, 1.0 mmol/mL, and 1.0 mmol/mL, respectively.

Then, 1 ml of a 0.001, glucose mmol/mL aqueous solution was added into the liquid composition, into which the evaluation reagent has been added, of each sample number.

As a result, hydrogen peroxide produced in the first stage of formula (1) progressed the reaction expressed in chemical formula (8) to produce a red quinone dye.

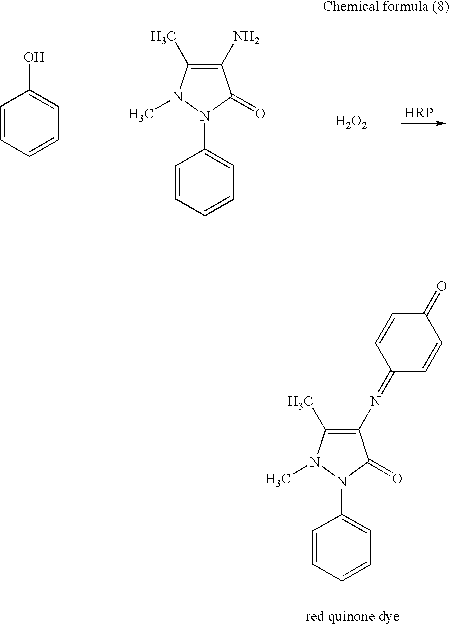

The produced amount of the red quinone dye was determined by measuring the absorbance at a wavelength of 505 nm. The resulting produced amount indirectly showed the activity of glucose oxidase (protein) contained in the liquid composition of each sample number.

The absorbance (activity) was measured 5 times on the liquid composition of each sample number.

The measured activity on each example was evaluated according to the following four levels based on the measured activity of glucose oxidase of the sample No. 6 as a reference.

Very good (VG): 0.80 times or more and 1.00 times or less with respect to the activity of sample No. 6.

Good (G): 0.60 times or more and less than 0.80 times with respect to the activity of sample No. 6.

Fair (F): 0.40 times or more and less than 0.60 times with respect to the activity of sample No. 6.

Poor (P): less than 0.40 times with respect to the activity of sample No. 6.

The results are shown in Table 1.

TABLE 1

| | Polyethylene glycol derivative | |
|---|---|---|
| Sample No. 1 | Chemical formula (3) | G |
| (present invention) | | |
| Sample No. 2 | Chemical formula (4) | G to VG |
| (present invention) | | |
| Sample No. 3 | Chemical formula (5) | G to VG |
| (present invention) | | |
| Sample No. 4 | Chemical formula (6) | VG |
| (present invention) | | |
| Sample No. 5 | Chemical formula (7) | P |
| (comparative example) | | |

As shown in Table 1, the result showed that the activities of glucose oxidase (protein) contained in the liquid compositions of the sample Nos. 1 to 4 (exemplary liquid compositions of the invention) were larger than the activity of the liquid composition of sample No. 5 (comparative example).

As a result, it was found that the lowering of the physiological activity (loss activity) of protein (enzyme) was preferably suppressed or prevented in the liquid compositions according to the invention.

The liquid compositions of sample Nos. 2 to 4 showed a tendency that the activity of glucose oxidase (protein) was larger than that of the liquid composition of the sample No. 1. Here, the sample Nos. 2 to 4 contained glucose oxidase (protein) joined the polyethylene glycol derivative, while the sample No. 1 contained glucose oxidase (protein) that was not joined the polyethylene glycol derivative.

The liquid composition of the sample No. 4 showed a nearly equal activity to that of the liquid composition of sample No. 6. Here, the weight-average molecular weight of the polyethylene glycol derivative was kept in an appropriate weight in the liquid composition of the sample No. 4. Therefore, it was found that little or nothing of the activity of glucose oxidase (protein) in the liquid composition was lost.

3-2. Evaluation on Sensors

As for the sensors made in the above examples and the comparative example, each sample store space was supplied with a 0.001 mmol/mL glucose aqueous solution (liquid sample). Then, a voltage was applied between the working electrode and the counter electrode of each sensor. Next, the maximum of the current (herein after, simply referred to as a current value) flowed between two electrodes was measured (μA).

In each example and the comparative example, the current value was measured on 5 sensors.

The current value was measured by applying a voltage of 0.5 V between the working electrode and the counter electrode.

As the result, all of the measured current values were larger than the current value measured on the comparative example. As for the four values, the sample No. 4 showed the highest, and the value was increasingly reduced in the sample Nos. 3 to 1 in order.

This result reflected the difference in the activity of glucose oxidase contained in the liquid composition of each sample measured in the evaluation on liquid compositions.

1. A method for forming a film, comprising:
preparing a liquid composition containing a substance for detecting an object and a polyether derivative having a weight-average molecular weight of 2000 or less;
supplying the liquid composition to a side adjacent to a surface of a base plate by using a droplet discharge method to form a liquid film; and
drying the liquid film to form a detection film containing the substance.

2. The method for forming a film according to claim 1,
wherein A divided by B is from 9 to 10⁵ where a content of the polyether derivative in the liquid composition is A wt % while a content of the substance in the liquid is B wt %.

3. The method for forming a film according to claim 1, wherein the liquid composition includes 10 wt % or less of the substance.

4. The method for forming a film according to claim 1, wherein the liquid composition includes 90 wt % or more of the polyether derivative.

5. The method for forming a film according to claim 1, wherein viscosity of the liquid composition is 20 cP or less at normal temperature.

6. The method for forming a film according to claim 1, wherein surface tension of the liquid composition is from 20 mN/m to 70 mN/m at normal temperature.

7. The method for forming a film according to claim 1, wherein a variation of the polyether derivative is within a range of ±0.2 C where C is a weight-average molecular weight of the polyether derivative.

8. The method for forming a film according to claim 1, wherein the polyether derivative includes a main chain made of polyether and a functional group that is provided at least one end of the main chain and bonds the substance.

9. The method for forming a film according to claim 8, wherein the main chain has the functional group at both ends thereof.

10. The method for forming a film according to claim 8, wherein the polyether derivative and the substance are joined by bonding the substance and the functional group in preparing the liquid composition.

11. The method for forming a film according to claim 1, wherein the polyether derivative mainly contains a polyethylene glycol derivative.

12. The method for forming a film according to claim 1, wherein the detection substance is protein.

13. The method for forming a film according to claim 12, wherein the protein is an enzyme.

14. A base plate with a film, comprising a detection film formed at the side adjacent to the surface of the base plate by the method for forming a film according to claim 1.

15. A base plate with a film, comprising a detection film containing a substance for detecting an object, wherein the detection film includes the substance and a polyether derivative having a weight-average molecular weight of 2000 or less.

16. A sensor, comprising the base plate with the film according to claim 14.

17. A liquid composition, comprising:
a substance having a physiological activity; and
a polyether derivative having a weight-average molecular weight of 2000 or less.
